(19) United States
(12) Patent Application Publication
(10) Pub. No.: US 2014/0186960 A1
(43) Pub. Date: Jul. 3, 2014

(54) Non Human Animal Models for Increased Retinal Vascular Permeability

(71) Applicant: Institut National De La Sante et De La Recherche Medicale (INSERM), Paris (FR)

(72) Inventors: Abdoulaye Sene, Paris (FR); Ramin Tadayoni, Paris (FR); Jose Sahel, Paris (FR); Alvaro Rendon, Paris (FR)

(73) Assignee: Institut National De La Sante et De La Recherche Medicale (INSERM), Paris (FR)

(21) Appl. No.: 14/174,963

(22) Filed: Feb. 7, 2014

Related U.S. Application Data
(62) Division of application No. 13/380,541, filed on Feb. 23, 2012.

(30) Foreign Application Priority Data
Jun. 26, 2009 (EP) 09305608.3

(51) Int. Cl. A01K 67/027; G01N 33/68

(52) U.S. Cl. CPC A01K 67/0276; G01N 33/6893
USPC 436/3

(57) ABSTRACT

The present invention relates to a non human animal model for increased retinal vascular permeability wherein said increased retinal vascular permeability is induced by inhibiting in Müller cells of said animal the expression of a gene encoding for Dp71 or a dystrophin associated protein (DAP). Furthermore, the present invention relates to methods and compositions for the treatment of a disease associated with an increased retinal vascular permeability in a subject in need thereof.

### FIELD OF THE INVENTION

The present invention relates to a non human animal model for increased retinal vascular permeability which could provide insight into the diagnosis and treatment of diseases associated with an increased retinal vascular permeability. Furthermore, the present invention relates to methods and compositions for the treatment of a disease associated with an increased retinal vascular permeability in a subject in need thereof.

### BACKGROUND OF THE INVENTION

Diabetic retinopathy (DR) is the leading cause of vision loss in working adults. Although its incidence and progression can be reduced by intensive glycemic and blood pressure control, nearly all patients with type I diabetes mellitus and over 60% of those with type 2 diabetes eventually develop retinal microvascular abnormalities, and 20% to 30% of these patients advance to active proliferative diabetic retinopathy (PDR) and/or diabetic macular edema. Although surgical options exist, developing preventative treatments for these disorders remains a major unmet clinical need.

Increased retinal vascular permeability (RVP) is a primary cause of diabetic macular edema and a characteristic finding in PDR.

Glial cells are crucially implicated in retinal function and integrity. Müller cells, the principal glia of the vertebrate retina are specialized radial glial cells which span the entire thickness of the retina and provide a wealth of functions that require an intimate interaction with the neurons and their synapses. Like astrocytes in the brain, Müller cells in the retina are crucial for tissue homeostasis, particularly in deactivating and recycling neurotransmitters and in maintaining the ionic balance of the extracellular fluid. Müller cells are also thought to participate in the induction, maintenance, and proper functioning of the blood-retina barrier (BRB).

In the neural retina, potassium buffering and water drainage via Müller cells are mediated by the cooperation of inwardly rectifying potassium channels (Kir), especially Kir4.1, with the selective water transport protein, Aquaporin-4 (AQP4). In healthy retina, Kir4.1 and AQP4 proteins are strongly expressed by Müller cells where these proteins are particularly enriched in the vitread endfeet and in cell processes abutting the intraretinal blood vessels. Proper functioning of Kir4.1 and AQP4 requires such a polarized expression of these channels in the plasma membrane of Müller cells.

The clustering and precise membrane localization of Kir4.1 and AQP4 are dependent on the dystrophin gene product, Dp71 (Kofuji P. et al. 2002; Dalloz C. et al. 2003). In the normal retina, the Dp71 protein shows the same expression pattern as Kir4.1 and AQP4; it has been shown to be involved in the targeting of these channels to specific membrane microdomains via a macromolecular protein complex composed by Dp71 and dystrophin-associated proteins (DAPs) (Dalloz C. et al. 2003; Fort P E. et al. 2008). Genetic inactivation of Dp71 (in Dp71-null mice) alters Kir4.1 and AQP4 distribution in Müller glial cells and this mislocation increases the vulnerability of retinal nerve cells to transient ischemia which is associated with neuronal cells death (Dalloz C. et al. 2003; Fort P E. et al. 2008). Together, these two types of glial channels play a key role in K+ and water balance processes; noteworthy, their expression and/or cytotopographical distribution are altered in many instances of retinal injuries, including transient ischemia reperfusion, diabetic retinopathy, retinal vein occlusion, and retinal detachment. Moreover, the early mislocation of Kir4.1 is accompanied by a dramatically decreased K+ conductance and a depolarization of the glial cell membrane which, in turn, impairs the function of the electrogenic uptake carriers for glutamate and GABA, subsequent neurotransmitter recycling, and other glia-neuron interactions. This may account for the occurrence of multiple and complex changes in neuronal and glial functions even before manifest vascular anomalies in diseases such as diabetic retinopathy can be detected, and suggests that the vascular pathology may be a consequence of disturbed glial cell functions. Previous work on mice had shown that the deletion of Dp71 and the concomitant impairments in DAPs localization cause functio-morphological alterations of Müller glial cells, particularly in the endfoot region where Dp71 and DAPs are concentrated in wildtype retinas (Fort P E. et al. 2008).

### SUMMARY OF THE INVENTION

The present invention relates to a non human animal model for increased retinal vascular permeability wherein said increased retinal vascular permeability is induced by inhibiting in Müller cells of said animal the expression of a gene encoding for Dp71 or a dystrophin associated protein (DAP).

### DETAILED DESCRIPTION OF THE INVENTION

The inventors applied experimental retinal detachment as a well-established model of retinal injury to both control (C57BL/6) and Dp71-null mice, and studied (i) the expression and cellular localization of the relevant proteins in Müller cells, (ii) the K+ currents across the Müller cell membrane, (iii) the swelling of Müller cell somata under osmotic challenge, and (iv) the retinal vascular permeability (i.e. permeability of the blood-retinal barrier). They found that experimental retinal detachment causes alterations in water transport and impairments in fluid absorption of Müller cells, associated with a downregulation of Dp71 expression. Similar functional deficits were observed in Müller cells from Dp71-null mice even without experimental retinal detachment; moreover, the retinal vascular permeability (i.e. permeability of the blood-retinal barrier) was dramatically increased already in untreated Dp71-null mice as compared to the controls. These results support the view that Dp71 and the DAPs, by controlling the expression and subcellular distribution of K+ and water channels, are essential for crucial glial cell functions such as retinal water homeostasis and maintenance of the blood retinal barrier.

### Non Human Model According to the Invention

The present invention relates to a non human animal model for increased retinal vascular permeability wherein said increased retinal vascular permeability is induced by inhibiting in Müller cells of said animal the expression of a gene encoding for Dp71 or a dystrophin associated protein (DAP).

The term “increased retinal vascular permeability” means that the retinal vascular permeability (i.e. permeability of the blood-retinal barrier) measured in said animal (e.g. mouse) is higher than the retinal vascular permeability generally observed in the general population of said animal (e.g. mouse). Typically, an increased retinal vascular permeability means that the retinal vascular permeability (i.e. permeability of the blood-retinal barrier) measured in said animal is least 1,5 fold, preferably 2 fold, more preferably 3 fold, still preferably 4 fold higher than the retinal vascular permeability generally observed in the general population of said animal (e.g. mouse). Quantification of retinal vascular permeability may be quantified any method well known in the art. For example, said retinal vascular permeability may be quantified by measuring albumin leakage from blood vessels into the retina using the Evans blue method as described by Xu Q. et al. (2001) or Tomasek J J. et al. (2006).

The term “Dp71” has its general meaning in the art and refers to the Dystrophin gene product 71. Dp71 protein consists of a unique seven-residues N-terminus fused to the cysteine-rich and C-terminal domains of dystrophin (Hugnot, J. P., (1992) Lederfein, D., (1992)). In addition, Dp71 transcripts can be alternatively spliced out for exons 71 and/or 78; while deletion of exon 71 does not change the reading frame, the loss of exon 78 does. This results in the replacement of the last 13 hydrophilic amino acids of dystrophin with 31 new hydrophobic amino acids in the Dp71 protein, which is called Dp71f isoform (Lederfein et al 1992; Rapaport et al 1992 and Kramarcy et al 1994). The term may include naturally occurring Dp71s and variants and modified forms thereof. The term may also refer to fusion proteins in which a domain from Dp71 that retains at least one Dp71 activity is fused, for example, to another polypeptide (e.g., a polypeptide tag such as are conventional in the art). The Dp71 can be from any source, but typically is a mammalian (e.g., human and non-human primate) Dp71, particularly a human Dp71. Exemplary native Dp71 amino acid and nucleotide sequences are depicted in table 1:

TABLE 1
isoforms of Dp71 transcript variants

| | Genebank | GenPept |
|---|---|---|
| Isoform | Accession Number | database Accc |
| Transcript variant Dp71ab, | NM_004018 | NP_004009 |
| mRNA | | |
| Transcript variant Dp71a, | NM_004017 | NP_004008 |
| mRNA | | |
| Transcript variant Dp71b, | NM_004016 | NP_004007 |
| mRNA | | |
| transcript variant Dp71, | NM_004015 | NP_004006 |
| mRNA | | |

The term “DAP” denotes a dystrophin associated protein. Dp71 represents the core of a multi-protein complex collectively termed the dystrophin-associated protein complex (DAPC). DAPs that comprise the DAPC are structurally organized into three distinct subcomplexes: the cytoskeletal proteins dystrophin, the dystrobrevins (α and β subunits) and the syntrophins (α, β and γ subunits); the dystroglycans (α and β subunits); and the sarcoglycans (α, β, γ, δ and ε subunits). In skeletal muscle, the DAPC is assembled around the dystrophin Dp427 this scaffold links the actin cytoskeleton to the basement membrane via the transmembrane protein β-dystroglycan and anchors the syntrophins and dystrobrevins to the muscle membrane (J. M. Ervasti (1991); O. Ibraghimov-Beskrovnaya, (1992)). In the retina, and more precisely in Muller glial cells the same dystrophin-associated protein complex implying Dp71 was characterized (Claudepierre T, et al. 2000).

In a particular embodiment, inhibiting in Müller cells of said animal the expression of a protein selected from the group of Dp71 protein or dystrophin associated proteins is performed with an inhibitor of expression.

An “inhibitor of expression” refers to a natural or synthetic compound that has a biological effect to inhibit or significantly reduce the expression of a gene.

Inhibitor of the expression may consist in a small organic molecule that inhibits the expression of Dp71 or DAP genes. In particular, inhibitors of Dp71 may consist in beta-nataphtoflavone as those described in Bermudez de Leon et al. (2006).

Inhibitors of expression for use in the present invention may be also based on anti-sense oligonucleotide constructs. Anti-sense oligonucleotides, including anti-sense RNA molecules and anti-sense DNA molecules, would act to directly block the translation of Dp71 or DAP mRNA by binding thereto and thus preventing protein translation or increasing mRNA degradation, thus decreasing the level of Dp71 and DAPs, and thus activity, in a cell. For example, antisense oligonucleotides of at least about 15 bases and complementary to unique regions of the mRNA transcript sequence encoding Dp71 or DAP can be synthesized, e.g., by conventional phosphodiester techniques and administered by e.g., intravenous injection or infusion. Methods for using antisense techniques for specifically inhibiting gene expression of genes whose sequence is known are well known in the art (e.g. sec U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of expression for use in the present invention. Dp71 or DAP gene expression can be reduced by contacting a subject or cell with a small double stranded RNA (dsRNA), or a vector or construct causing the production of a small double stranded RNA, such that Dp71 or DAP gene expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al, (2002); Brummelkamp, T R. et al. (2002); U.S. Pat. Nos. 6,573,099 and 6,506,559; and International Patent Publication Nos. WO 01/36646, WO 99/32619, and WO 01/68836). All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNAs sequences advantageously comprises at least twelve contiguous dinucleotides or their derivatives.

As used herein, the term “siRNA derivatives” with respect to the present nucleic acid sequences refers to a nucleic acid having a percentage of identity of at least 90% with erythropoietin or fragment thereof, preferably of at least 95%, as an example of at least 98%, and more preferably of at least 98%.

As used herein, “percentage of identity” between two nucleic acid sequences, means the percentage of identical nucleic acid, between the two sequences to be compared, obtained with the best alignment of said sequences, this percentage being purely statistical and the differences between these two sequences being randomly spread over the nucleic acid acids sequences. As used herein, “best alignment” or “optimal alignment”, means the alignment for which the determined percentage of identity (see below) is the highest. Sequences comparison between two nucleic acids sequences are usually realized by comparing these sequences that have been previously align according to the best alignment; this comparison is realized on segments of comparison in order to identify and compared the local regions of similarity. The best sequences alignment to perform comparison can be realized, beside by a manual way, by using the global homology algorithm developed by SMITH and WATERMAN (Ad. App. Math., vol. 2, p:482, 1981), by using the local homology algorithm developed by NEDDLEMAN and WUNSCH (J. Mol. Biol., vol. 48, p:443, 1970), by using the method of similarities developed by PEARSON and LIPMAN (Proc. Natl. Acd. Sci. USA, vol. 85, p:2444, 1988), by using computer softwares using such algorithms (GAP, BESTFIT, BLAST P, BLAST N, PASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p:1792, 2004). To get the best local alignment, one can preferably used BLAST software. The identity percentage between two sequences of nucleic acids is determined by comparing these two sequences optimally aligned, the nucleic acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

shRNAs (short hairpin RNA) can also function as inhibitors of expression for use in the present invention.

Ribozymes can also function as inhibitors of expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucicolytic cleavage. Engineered hairpin or hammerhead motif ribozyme molecules that specifically and efficiently catalyze endonucleolytic cleavage of Dp71 or DAP mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of expression can be prepared by known methods. These include techniques for chemical synthesis such as, e.g., by solid phase phosphoramidite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing Dp71 or DAP. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adenoviruses and adeno-associated (AAV) viruses, which are double-stranded DNA viruses that have already been approved for human use in gene therapy. Actually 12 different AAV serotypes (AAV1 to 12) are known, each with different tissue tropisms (Wu, Z Mol Ther 2006; 14:316-27). Recombinant AAV are derived from the dependent parvovirus AAV2 (Choi, V W J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989, In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBluescript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

Alternatively, the animal may be deficient for a gene or a plurality of genes so that said animal cannot produce Dp71 or DAPs. For example, said animal may be deficient in a gene encoding for Dp71 or a DAP.

Deficient animals, especially deficient mice are generally commercially available. However, methods for producing deficient animal or knock-out animal are well known in the art. For example, Knock-out animals comprising targeted mutations are achieved routinely in the art as provided for example by the method by Joyner, A. L. (Gene Targeting. 1999, Second Edition. The Practical Approach Series, Oxford University Press, New York) and Hogan, B., et al. (Manipulating the mouse embryo. 1994, Second Edition, Cold Spring Harbor Press, Cold Spring Harbor.). For example, the heterozygous and/or homozygous knock-out animal of the above-described methods may be generated by selecting embryonic stem (ES) cell clones carrying a mutated allele for the gene of interest, verifying the targeted mutation in the recombinant embryonic stem cell clones, injecting the verified recombinant embryonic stem cells into blastocysts of wild type animals, transferring these injected blastocysts into pseudo-pregnant foster mothers, breeding chimeras resulting from the blastocysts to wild type animals, testing the offspring resulting from these breedings for the presence of the targeted mutation, breeding heterozygous animals, optionally to generate homozygous knock-out animals. Embryonic stem cells used in the art which may also be used in the methods of this invention comprise for example embryonic stem cells derived from mouse strains such as C57BL/6, BALB/c, DBA/2, CBA/and SV129. Preferably, embryonic stem cells derived from C57BL/6 mice are used (Scong, E et al (2004) Trends Genet. 20, 59-62; Wolfer, D. P. et al, Trends Neurosci. 25 (2002): 336-340). Methods for producing deficient animals are also described in WO03037074 and US2007056052.

Alternatively, the knock out of the targeted gene may be performed in conditional manner as described in WO2006048466 or WO2006048465, so that the disruption of expression may be performed at a willing time and/or in a specific tissue or cells such as Müller cells.

For example a deficient animal for Dp71 may be obtained according to the method disclosed in EXAMPLE and as described by Sarig R, et al. (1999).

The animal according to the invention may be selected from the group consisting of rat, mouse, cow, pig, horse, chicken, cat, and dog. More preferably, the animal is a mouse. Said mouse may be selected from the groups consisting of C57BL/6, BALB/c, DBA/2, CBA/and SV129 strains.

A further aspect of the invention relates to use of a non human deficient animal for Dp71 as a model for increased retinal vascular permeability.

### Uses of the Non Human Model According to the Invention

Such animal model may be useful for screening drugs useful for reducing increased retinal vascular permeability. More particularly, the animal model according to the invention may be suitable for screening drugs for treating a disease associated with an increased retinal permeability, studying the physiopathology of said disease, and for endeavouring diagnostic or prognostic markers of said disease.

As used herein, the term “disease associated with an increased retinal permeability” includes but is not limited to the diseases selected from the group consisting of retinal injuries such as transient ischemic/reperfusion, diabetic retinopathy, retinal vein occlusion, macula oedema, retinal detachment, and age-related retinal degeneration.

A further aspect of the invention relates to a method for screening a drug for treating a disease associated with an increased retinal vascular permeability, said method comprising the step consisting of:

i) providing a non human animal model for increased retinal vascular permeability according to the invention

ii) administering to said animal a candidate compound

iii) measuring the effect of said compound on the blood-retinal barrier integrity of said animal.

iv) selecting the candidate compound capable of restoring the blood-retinal barrier integrity in said animal.

Quantification of blood-retinal barrier integrity may be quantified by measuring albumin leakage from blood vessels into the retina using the Evans blue method as described by Xu Q. et al. (2001); Tomasek J J. et al. (2006) and in EXAMPLE.

According to a one embodiment of the invention, the candidate compound of may be selected from a library of compounds previously synthesised, or a library of compounds for which the structure is determined in a database, or from a library of compounds that have been synthesised de novo. The candidate compound may be selected from the group of (a) proteins or peptides, (b) nucleic acids and (c) organic or chemical compounds. Illustratively, libraries of pre-selected candidate nucleic acids may be obtained by performing the SELEX method as described in documents U.S. Pat. No. 5,475,096 and U.S. Pat. No. 5,270,163.

### Diagnostics Methods According to the Invention

A further aspect of the invention relates to a method of testing a subject thought to have or be predisposed to having a disease associated with an increased retinal vascular permeability, which comprises the step of analyzing a biological sample from said subject for:

(i) detecting the presence of a mutation in the gene encoding for Dp71 or a DAP and/or its associated promoter, and/or

(ii) analyzing the expression of the gene encoding for Dp71 or a DAP.

As used herein, the term “subject” denotes a mammal, such as a rodent, a feline, a canine, and a primate. Preferably, a subject according to the invention is a human. The term “subject in need thereof” is intended for a subject affected or likely to be affected with a disease associated with an increased retinal vascular permeability.

As used herein, the term “biological sample” refers to any sample from a subject such as blood or serum.

Typical techniques for detecting, a mutation in the gene encoding for Dp71 or a DAP may include restriction fragment length polymorphism, hybridisation techniques, DNA sequencing, exonuclease resistance, microsequencing, solid phase extension using ddNTPs, extension in solution using ddNTPs, oligonucleotide assays, methods for detecting single nucleotide polymorphism such as dynamic allele-specific hybridisation, ligation chain reaction, mini-sequencing, DNA “chips”, allele-specific oligonucleotide hybridisation with single or dual-labelled probes merged with PCR or with molecular beacons, and others.

Analyzing the expression of the gene encoding for Dp71 or a DAP may be assessed by any of a wide variety of well-known methods for detecting expression of a transcribed nucleic acid or translated protein.

In a preferred embodiment, the expression of the gene encoding for Dp71 or a DAP is assessed by analyzing the expression of mRNA transcript or mRNA precursors, such as nascent RNA, of said gene. Said analysis can be assessed by preparing mRNA/cDNA from cells in a biological sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFF YMETRIX).

Advantageously, the analysis of the expression level of mRNA transcribed from the gene encoding for Dp71 or a DAP involves the process of nucleic acid amplification, e.g., by RT-PCR (the experimental embodiment set forth in U.S. Pat. No. 4,683,202), ligase chain reaction (BARANY, Proc. Natl. Acad. Sci. USA, vol. 88, p: 189-193, 1991), self sustained sequence replication (GUATELLI et al., Proc. Natl. Acad. Sci. USA, vol. 57, p: 1874-1878, 1990), transcriptional amplification system (KWOH et al., 1989, Proc. Natl. Acad. Sci. USA, vol. 86, p: 1173-1177, 1989), Q-Beta Replicase (LIZARDI et al., Biol. Technology, vol. 6, p: 1197, 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In another preferred embodiment, the expression of the gene encoding for Dp71 or a DAP is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair biotin-streptavidin)), or an antibody fragment (e.g., a single-chain antibody, an isolated antibody hypervariable domain, etc.) which binds specifically to the protein translated from the gene encoding for Dp71 or a DAP.

Said analysis can be assessed by a variety of techniques well known from one of skill in the art including, but not limited to, enzyme immunoassay (EIA), radioimmunoassay (RIA), Western blot analysis and enzyme linked immunoabsorbent assay (RIA).

The method of the invention may comprise comparing the level of expression of the gene encoding for Dp71 or a DAP in a biological sample from a subject with the normal expression level of said gene in a control. A significantly weaker level of expression of said gene in the biological sample of a subject as compared to the normal expression level is an indication that the patient has or is predisposed to developing a disease associated with an increased retinal vascular permeability. The “normal” level of expression of the gene encoding for Dp71 or a DAP is the level of expression of said gene in a biological sample of a subject not afflicted by any disease associated with an increased retinal vascular permeability. Preferably, said normal level of expression is assessed in a control sample (e.g., sample from a healthy subject, which is not afflicted by any disease associated with an increased retinal vascular permeability) and preferably, the average expression level of said gene in several control samples.

### Therapeutic Methods and Pharmaceutical Compositions According to the Invention

A further aspect of the invention relates to methods, compositions (such as pharmaceutical compositions) and uses for treating a disease associated with an increased retinal vascular permeability.

In its broadest meaning, the term “treating” or “treatment” refers to reversing, alleviating, inhibiting the progress of, or preventing the disorder or condition to which such term applies, or one or more symptoms of such disorder or condition.

Thus, a further aspect of the invention relates to a polypeptide selected from the group consisting of Dp71 polypeptide, a DAP or a variant thereof for use in the treatment of a disease associated with an increased retinal vascular permeability.

Dp71 polypeptides, DAPs or a variants thereof may be produced by any technique known per se in the art, such as without limitation, any chemical, biological, genetic or enzymatic technique, either alone or in combination(s). Preferred techniques include the expression in any appropriate host cell of a corresponding coding nucleic acid molecule or the artificial synthesis using conventional techniques such as solid phase synthesis.

For use in the present invention, the polypeptide may be used in isolated (e.g., purified) form or contained in a vector, such as a membrane or lipid vesicle (e.g. a liposome).

Alternatively, a nucleic acid construct encoding for a Dp71 polypeptide, a DAP or a variant thereof may be used.

Therefore the same methods as above described for antisense oligonucleotide, siRNA, shRNA or ribozyme nucleic acid sequence may be used for delivering the nucleic acid construct encoding for said Dp71 polypeptide, a DAP or a variant thereof.

Said above vectors or constructs may also be used to produce a Dp71 polypeptide, a DAP or a variant thereof in vitro or ex vivo, upon introduction into a suitable host cell. Examples of such cells include, for instance, mammalian, yeast, plant, insect or bacterial cells, such as primary mammalian cells or established cell line cultures. Specific examples of mammalian cells include PC 12 cells. Amongst bacterial and yeast cells, E. coli, Saccharomyces and Kluyveromyces cells may be cited.

A further aspect of the invention relates to an activator of expression of a Dp71 polypeptide, a DAP or a variant thereof for use in the treatment of a disease associated with an increased retinal vascular permeability.

The invention also provides a method for treating a disease associated with an increased retinal vascular permeability comprising administering a subject in need thereof with a therapeutically effective amount of a Dp71 polypeptide, a DAP or a variant thereof (or a nucleic acid construct encoding for a Dp71 polypeptide, a DAP or a variant thereof according to the invention.

The compounds of the invention, such as Dp71 polypeptide or variants thereof may be combined with pharmaceutically acceptable excipients, and optionally sustained-release matrices, such as biodegradable polymers, to form therapeutic compositions.

The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating, action of microorganisms, such as bacteria and Fungi.

Solutions comprising compounds of the invention as Free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The inhibitor of the invention can be Formulated into a composition in a neutral or salt form. Pharmaceutically acceptable salts include the acid addition salts (Formed with the free amino groups of the protein) and which are Formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The compounds of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The compounds of the invention may be delivered in a pharmaceutically acceptable ophthalmic vehicle, such that the polypeptide can penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically-acceptable ophthalmic vehicle may, for example, be an ointment, vegetable oil or an encapsulating material. Alternatively, inhibitors of the invention may be injected directly into the vitreous, aqueous humour, ciliary body tissue(s) or cells and/or extra-ocular muscles by electroporation means.

The invention will be further illustrated by the following figures and examples. However, these examples and figures should not be interpreted in any way as limiting the scope of the present invention.

### FIGURES

FIG. 1. Dramatic reduction of Dystrophin Dp71 expression in detached retina: A: Western blot analysis of Dp71 and β-actin total protein levels in control and detached retina 24 h and 7 days after experimental retinal detachment. Approximate molecular masses are given on the left of each panel in kiloDaltons. B: Dp71 and β-actin expression were quantified by densitometry analysis of western blots bands using TotalLab TL120 software (Nonlinear Inc, Durham N.C., USA). The bar graph shows Dp71/β-actin ratio. In detached retina, Dp71 protein level is dramatically reduced after 24 h and this decrease remains 7 days after surgery. C: The mRNA expression of Dp71 was determined by real-time PCR in control and detached retina at 24 h and 7 days after surgery and was normalized to the levels of mRNA for β-actin. The Dp71 mRNA level in detached retina was strongly reduced. Dara are expressed as mean±SE; n=4. **p<0.01; ***p<0.001 significant differences versus control.

FIG. 2. Retinal detachment reduced potassium current density of murine Müller glial cells. The cells were isolated from control and detached retina of wt and Dp71-null mice and were investigated 7 days after surgery. Current density was calculated from the inward current recorded at a hyperpolarizing step from −80 to −140 mV normalized to cell membrane capacitance. Current densities decreased in detached retina from wt and Dp71-null mice. Each bar represents values obtained in 12 cells from 3 mice. Data are expressed as mean±SD. **p<0.01; significant differences versus control.

FIG. 3. Osmotic swelling properties of Müller glial cells. Müller cell somata were measured after 4 minutes perfusion of retinal slices with a hypotonic solution (60% of control osmolarity). Data were expressed in percentage of the values obtained before hypotonic challenge. A: Müller cells of control and detached retina from wt and Dp71-null mice were exposed to hypotonic challenge. In wt mice, only cells of detached retina swell. Note the swelling of Müller cells from control retina of Dp71-null mice, B: Müller cells of control retina from wt and Dp71-null mice were exposed to hypotonic challenge in the presence of barium chloride (Ba²⁺). Addition of Ba²⁺ induced a swelling of Müller cells of wt mice and had no effect on the soma area of Müller cells of Dp71-null mice. C: Addition of adenosine (aden); inhibitor of phospholipase A₂, 4-bromophenacyl bromide (from) and inhibitor of cyclooxygenase enzyme, indomethacin (indo) blocked the swelling of Müller cells of control retina from Dp71-null mice. Each bar represents value obtained in 12-34 cells; n=3 mice for each group. Data are expressed as mean+SE. **p<0.01; ***p<0.001 significant differences versus control. ^{∘∘∘}p<0.001 significant difference versus wt.

FIG. 4. Implication of Dp71 in blood-retinal barrier permeability. Blood-retinal barrier permeability was measured using Evans blue dye vascular permeability assay on wt and Dp71-null adult mice. Vascular permeability was strongly increased in Dp71-null mice compared with C57BL/6 mice. Data are expressed as mean±SE; n=4. *p<0.05; significant differences versus control.

### EXAMPLE

### Material & Methods

Animals:

The Dp71-null mice (Sarig R. et al. 1999) were obtained by replacing, via homologous recombination, most of the first and unique exon of Dp71 and of a small part of Dp71 first intron with a sequence encoding a β-gal-neomycine-resistance chimeric protein (β-geo). This abolished the expression of Dp71 without interfering with the expression of other products of the DMD (Duchenne Muscular Dystrophy) gene. C57BL/6J mice strain (Charles River, France) was used as controls For this study. All animal use was conducted in accordance with the guidelines of the Association for Research in Vision and Ophthalmology (ARVO) Statement for the Use of Animals.

Antibodies:

Monoclonal antibodies targeting β-Actin and GFAP were purchased from Sigma-Aldrich (Deisenhofen, Germany). Polyclonal antibodies directed against dystrophins (H4) and utrophin (K7) were previously characterized (Rivier F. et al. 1999), whereas the ones directed against Kir4.1 and AQP4 were from Alomone Labs (Souffelweyersheim, France).

Induction of Retinal Detachment:

Local retinal detachment was induced in the right eye of adult mice, as previously reported with minor modification (Yang L. et al. 2004). Briefly, mice were anesthetized by peritoneal injection of a mixture of 62.5 mg/kg ketamine (Virbac, France) and 12.5 mg/kg xylazine (Bayer, France). The pupils of the eyes were dilated by topical tropicamide 0.5% (Thea, France) and retinal detachment was created by subretinal injection of sodium hyaluronate. The left eye served as control. Animals with lens injury, vitreous hemorrhage and eye infection were excluded from this study.

Immunohistochemistry:

Enucleated eyes were dissected to remove lens and cornea, and fixed by immersion in 4% paraformaldehyde for 1 h. Fixed eyes were cryoprotected in 30% sucrose, frozen and embedded in Cryomatrix (Thermo Shandon, Pittsburgh, Pa., United States), and then cut into 10 μm cryostat sections mounted on SuperFrost/Plus slides (0. Kindler, Freiburg, Germany). Sections were permeabilized for 10 minutes with 0.1% PBS (Phosphate Buffer Saline)-Triton X100 and blocked for 1 h with 0.1% normal goat serum, 3% bovine serum albumin, 0.1% Tween 20, and 1×PBS. Sections were then incubated with primary antibody for 2 h at room temperature. After several washes with PBS, secondary antibodies (Interchim, France) coupled to Alexa were used diluted 1:500 in 0.5% bovine scrum albumin for 1 h. Sections were rinsed, mounted with Fluorsave reagent (Calbiochem, San Diego, Calif., USA) and viewed with a DM 5000B microscope (Leica Microsystems SAS, Rucil Malmaison, France) equipped with a Photometrics cool SNAP TM FX camera (Ropper Scientific, Tuscan, Ariz., USA).

Western Blot Analysis:

Western blot analysis was performed as previously described (Bordais A. et al. 2005). In brief, retinal protein extracts were resolved using NuPAGE Tris-Acetate 3-8% gradient gels (Invitrogen) and electrotransferred to polyvinylidene difluoride (PVDF) membranes according to the manufacturer's instructions. PVDF membranes were blocked in PBS containing 0.1% Tween 20, 1% BSA, 5% dry milk (BIO-RAD, Herts, UK) for 1 h at room temperature then incubated with the primary antibody in the same blocking buffer. Blots were then washed and incubated secondary antibody conjugated to horseradish peroxidase (Jackson Immunoresearch laboratories). Chemiluminescence was performed using ECL plus Western blotting detection system (GE Healthcare, Germany) and documented on film (GE Healthcare, Germany).

Quantitative RT-PCR Analysis of Retinal RNA:

Total RNA from retina was extracted using Trizol reagent (Invitrogen, France) according to the manufacturer's instructions. Reverse transcription was performed on 1 μg total RNA using SuperScript II and random hexamers (Invitrogen, France). PCR amplifications of cDNA were performed using Master plus SYBR Green I (Roche Diagnostics, Germany) on a LightCycler instrument (Roche Products, Basel, Switzerland). PCR primers were designed using Primer3 software. For relative comparison, the C₁ values of real-time PCR results were analysed using the ΔC₁ method according to the manufacturer's instructions. The amount of Dp71 cDNA was normalized to the standard internal control obtained using primers for β-actin.

Glial Cell Isolation:

The retinae were isolated and special care was taken to separate detached areas. Retinal pieces were incubated in papain (0.2 mg/ml; Roche Molecular Biochemicals, Mannheim, Germany)-containing Ca²⁺- and Mg²⁺-free phosphate-buffered saline, pH 7.4, for 30 minutes at 37° C., followed by several washing steps with saline. After short incubation in saline supplemented with DNase I (200 U/ml; Sigma, Taulkirchen, Germany) the tissue pieces were triturated by a pipette, to obtain suspensions of isolated cells. The cells were stored at 4° C. in serum-free minimum essential medium (Sigma) until use within three hours after isolation.

Electrophysiological Recordings:

Membrane currents of acutely isolated glial cells were recorded in the whole-cell configuration of the patch-clamp technique. Voltage-clamp records were performed at room temperature (22-25 CC) using the Axopatch 200A amplifier (Axon Instruments. Foster City, Calif.) and the ISO-2 computer program (MFK, Niedernhausen, Germany). The signals were low-pass filtered at 1, 2, or 6 kHz (eight-pole Bessel filter) and digitized at 5, 10, or 30 kHz, respectively, using a 12-bit A/D converter. Patch pipettes were pulled from borosilicate glass (Science Products, Hofheim, Germany) and had resistances between 4 and 6 MΩ when filled with a solution containing (mM) 10 NaCl, 130 KCl, 1 CaCl₂, 2 MgCl₂, 10 ethyleneglycolbis(aminoethylether)tetra-acetate, and 10 N-2-hydroxyethyl-piperazine-N′-2-ethanesulphonic acid (HEPES) adjusted to pH 7.1 with Tris-base. The recording chamber was continuously perfused with extracellular solution which contained (mM) 135 NaCl, 3 KCl, 2 CaCl₂. 1 MgCl₄, 1 Na₂HPO₄, 10 HEPES, and 11 glucose; the solution was equilibrated to pH 7.4 with Tris-base.

To evoke membrane currents, dc- and hyperpolarizing voltage steps of 250 ms duration, with increments of 10 mV, were applied from a holding potential of −80 mV. The amplitudes of the steady-state inward currents were measured at the end of the voltage step from −80 to −140 mV. To activate transient voltage-dependent currents, a hyperpolarizing prepulse (500 ms, −120 mV) was applied before depolarization. The membrane capacitance of the cells was measured by the integral of the uncompensated capacitive artefact (filtered at 6 kHz) evoked by a hyperpolarizing voltage step from −80 to −90 mV when Ba²⁺ ions (1 mM) were present in the bath solution to block the K⁺ conductance. Membrane potentials were measured in the current-clamp mode. Statistical analysis (unpaired Student's t test) was performed using the SigmaPlot program (Jandel Corp.); data are expressed as means±standard deviation. Cells from the operated and the contralateral control eyes from 3 C57BL/6 and 3 Dp71-null mice were used.

MüLler Cell Swelling:

To determine volume changes of Müller glial cells evoked by hypotonic stress, the cross-sectional area of glial cell somata in the inner nuclear layer of retinal slices was measured. Acutely isolated slices (thickness, 1 mm) were prepared from untreated control eyes and from the detached retinal area of operated eyes from C57BL/6 and Dp71-null mice, placed in a perfusion chamber and loaded with the vital dye Mitotracker Orange (10 μM). This dye is selectively taken up by Müller glial cells whereas retinal neurons, astrocytes, and microglial cells remain unstained [46]. The stock solution of the dye was prepared in dimethylsulfoxide and resolved in saline. The slices were examined with a confocal laser scanning microscope LSM 510 Meta (Zeiss, Obcrkochen, Germany). Mitotracker Orange was excited at 543 nm, and emission was recorded with a 560 nm long-pass filter. To assure that the maximum soma areas were precisely recorded, the focal plane was continuously adjusted in the course of the experiments.

A gravity-fed system with multiple reservoirs was used to perfuse the recording chamber continuously with extracellular solution; the hypotonic solution and test substances were added by rapid change of the perfusate. The extracellular solution consisted of (in mM) 136 NaCl, 3 KCl, 2 CaCl₂, 1 MgCl₂, 10 HEPES, and 11 glucose, adjusted to pH 7.4 with Tris. The hypotonic solution (60% of control osmolarity) was made up by adding distilled water. BaCl₂ (1 mM) was preincubated for 10 minutes in extracellular solution before application within hypotonic solution. Blocking substances were preincubated for 15 to 45 minutes, and adenosine was administered simultaneously with the hypotonic solution. Swelling experiments were performed at room temperature.

To determine the extent of glial soma swelling, the cross-sectional area of Mitotracker Orange-stained cell bodies in the inner nuclear layer of retinal slices was measured off-line using the image analysis software of the LSM. Bar diagrams display the mean (+SEM) cross-sectional area of cell somata that was measured after 4-minute perfusion with hypotonic solution, in percent of the soma area measured before osmotic challenge (100%).

Quantification of Blood-Retinal Barrier Permeability:

Vascular permeability was quantified by measuring albumin leakage from blood vessels into the retina using the Evans blue method [32]. Briefly, mice were anesthetized and Evans blue (45 mg/kg; Sigma-Aldrich, Germany) was injected through the jugular vein. Blood samples were taken 3 h after injection of the dye and mice were perfused for 2 minutes via the left ventricle with a citrate buffer (0.05 M, pH 3.5) prewarmed to 37° C. After perfusion, both eyes were enucleated and carefully dissected. Retinas were dried in a Speed-Vac for 5 h, weighed and the Evans blue dye was extracted by incubating the retina with 100 μl of formamide for 18 h at 70° C. Retina samples were centrifuged at 70,000 rpm for 20 minutes and blood samples were centrifuged at 12,000 rpm for 15 minutes. Both supernatants were used to measure absorbance. A background-subtracted absorbance was determined by measuring each sample at both 620 nm, the absorbance maximum for Evans blue, and 740 nm, the absorbance minimum. Evans blue concentration in the plasma and the retina was calculated from a standard curve of Evans blue in formamide. Blood-retinal barrier permeability was expressed in microliter of Evans blue per gram of wet retina per hour (μl Evans blue×g wet retina⁻¹×h⁻¹).

Data Analysis:

Statistical significance of data obtained from Western blot, RT-PCR and electrophysiological recording was determined by unpaired Student's t-test with the use of Prism 5 (GraphPad Software, San Diego, Calif.). To determine the extent of Müller cells soma swelling, significance analysis was made using Mann-Whitney U-test for two groups and analysis of variance followed by comparisons for multiple groups.

### Results

Experimental Retinal Detachment Induces Reactive Gliosis and Alterations in Protein Expression in MüLler Cells:

To prove the induction of reactive Müller cell gliosis by experimental detachment, the expression of glial fibrillary acidic protein (GFAP) was assessed. In control retinas, GFAP was only localized in astrocytes, at the inner retinal surface. Experimental retinal detachment caused an unequivocal upregulation of GFAP expression in Müller glial cells. Twenty-four hours after surgery, Western blot analysis confirmed an increased level of GFAP protein expression. For a semi-quantitative analysis, immunoblot band densities were quantified and normalized to β-Actin. This showed that in the detached retina, the GFAP level was elevated by a factor of 3.

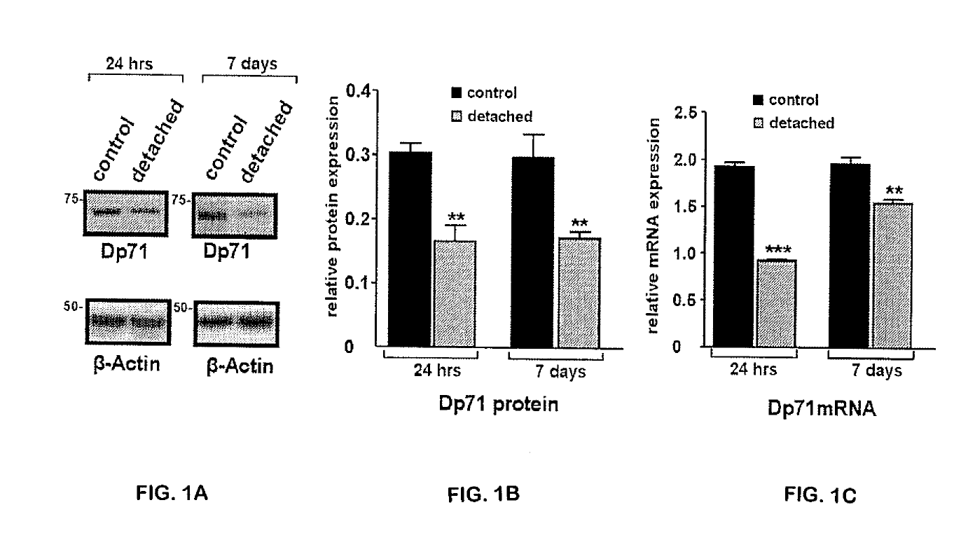

Immunohistochemistry demonstrated unequivocal Dp71 immunoreactivity mainly in the endfeet of Müller cells and around blood vessels of control retinas, as previously reported (Dalloz C. et al. 2003) whereas immunolabeling was considerably reduced in detached retinas. To determine whether retinal detachment altered the protein and mRNA expression of Dp71 in C57BL/6 mice, we performed Western blot and real-time PCR analysis at 24 hours and 7 days after surgery. By using Western blotting and subsequent semiquantification of band densities of Dp71, we observed a dramatic reduction of Dp71 protein in detached retinas (FIG. 1A), by about 50% (FIG. 1B). Furthermore, we found that the Dp71 mRNA level in detached retinas was 50% lower than in control retinas, 24 hours after surgery. After 7 days, the Dp71 mRNA appeared to recover, as it was reduced by only 20% if compared to the controls. Together these data show that experimental retinal detachment-induced gliosis affects both gene and protein expression of Dp71 in Müller glial cells.

Kir4.1, the principal K⁺ channel in retina, was localized in the endfeet of Müller cells at the inner limiting membrane and around blood vessels of control retinas, as previously described. In detached retinas, Kir4.1 was distributed all along the Müller cells fibers. However, Kir4.1 protein levels were not changed in detached retinas. Thus, retinal detachment induced a loss of the polarized expression of Kir4.1 but this mislocalization of Kir4.1 was not associated to a decrease of its expression level.

AQP4, the predominant aquaporin channel in retina, was found to be enriched at the endfeet of Müller cells, around blood vessels and in the open plexiform layer (, similar as previously reported. Twenty-four hours of retinal detachment, the overall pattern of AQP4 distribution was unchanged but the immunolabeling intensity was decreased. This observation was confirmed by immunoblot analysis of AQP4 expression, revealing a significant decrease of the AQP4 expression level by 30% as compared to the controls.

Earlier it had been shown that utrophin, a cytoskeleton protein, shares the common expression pattern of Dp71, Kir4.1, and AQP4, and that Dp71 deletion induces an upregulation of utrophin in retinas and isolated Müller cells of mice (Dalloz C. et al. 2003), probably via its interaction with Kir4.1 and AQP4 in a protein complex involving the DAPs. To further characterize utrophin localization and expression in detached retina, we performed immunohistochemistry. In control retinas, utrophin was localized around blood vessels and in the inner limiting membrane. Retinal detachment increased the intensity of utrophin immunolabeling.

Summarizing these results, both gene and protein expression of Dp71 are strongly reduced after experimental retinal detachment in C57BL/6 mice. This may be responsible for the mislocation of Kir4.1 and the downregulation of AQP4 (as well as for a compensatory upregulation of utrophin). To test this hypothesis, experimental retinal detachment was performed in the eyes of adult Dp71-null mice. As previously reported (Dalloz C. et al. 2003; Fort P E. et al. 2008), Kir4.1 and AQP4 were already mislocalized/downregulated in untreated Dp71-null mice, and retinal detachment failed to induce any additional alterations of these channels at 24 h or 7 days after surgery.

Retinal Detachment Causes Morphological and Physiological Alterations in C57BL/6 and Dp71-Null Mice:

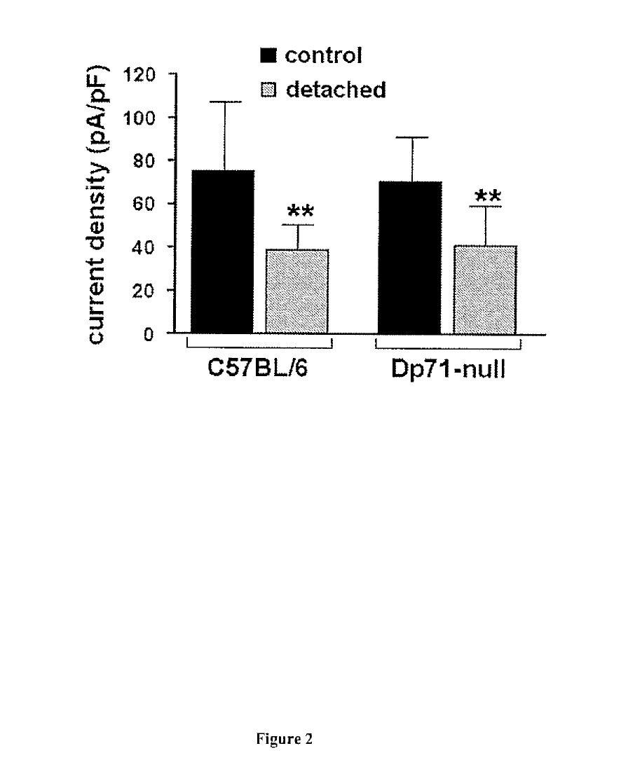

To test whether the observed mislocation of Kir4.1 altered the K⁺ currents of Müller dial cells as described in other animal models, we recorded membrane currents in dissociated Müller cells of control and detached retina from C57BL/6 and Dp71-null mice. In our model of retinal detachment in C57BL/6 mice, a decrease of K⁺ current amplitudes was observed although it was less prominent (by about 25%) than reported in rabbit and pig detachment models. Müller cells from detached retinal areas of Dp71-null mice did not show a significant reduction of the inward currents. We tested now whether the current densities (i.e., current amplitude divided by membrane capacitance) might have been altered, as reactive Müller cells usually are hypertrophic and display an increased membrane area. The membrane capacitance of Müller cells, as an indicator of the membrane area, can be recorded when the dominating conductance is blocked by 1 mM Ba²⁺. There was no significant difference between the capacitances of Müller cells from untreated eyes of C57BL/6 mice (40±12 pF, n=12) and those of cells from untreated eyes of Dp71-null mice (41±8 pF, n=11). However, Müller cells from the detached retinas of the contralateral operated eyes displayed a significantly increased membrane capacitance in both strains (C57BL/6:52±10 pF, P<0.01; Dp71-null: 69±20 pF, n=12, P<0.001), suggesting a cellular hypertrophy. There was the possibility that the cell surface increased without insertion of new channel proteins into the membrane. To test this possibility, we calculated the K⁻ current densities. Indeed we found that experimental retinal detachment evoked a significant (p<0.01) decrease of the inward current densities of Müller glial cells from both mice strains (controls, from 75.1±32.0 to 38.8±11.6 pA/pF; Dp71-null, from 70.6±20.3 to 41.1±18.2 pA/pF) (FIG. 2).

Other parameters of Müller cell electrophysiology (membrane potential, outward current amplitude, incidence of cells expressing fast voltage-dependent Na⁺ currents) did not show significant differences between control and detached retinas from both strains of mice.

Osmotic MüLler Cell Swelling of C57BL/6 Injured and Dp71-Null Retinae:

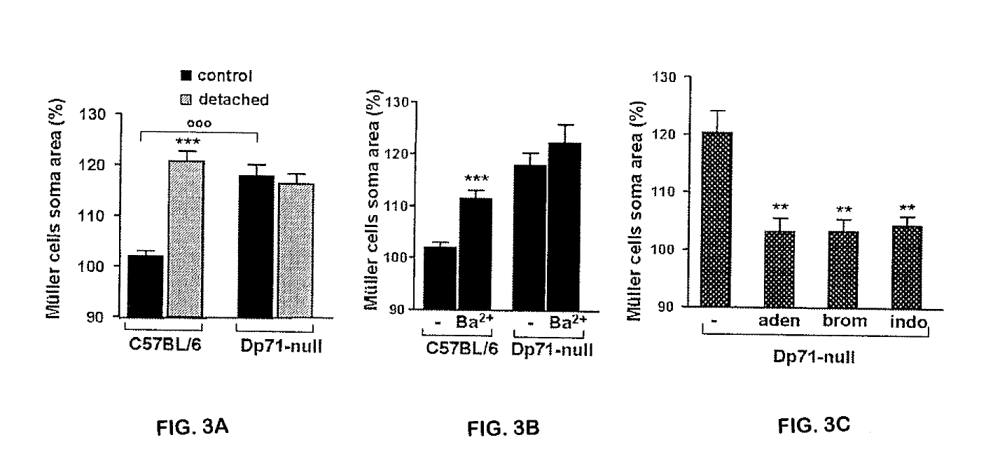

Previous work has revealed that reactive Müller cells may display alterations in transmembranous water transport. Thus, the swelling of Müller cell somata was investigated in acutely isolated retinal slices by perfusing the slices with a hypotonic solution containing 60% of control osmolarity. Exposure to hypotonic solution did not alter the size of Müller cell bodies in retinal slices from untreated eyes of C57BL/6 mice (102.0±1.0%, n=34). However, a 4-min perfusion of slices from detached retinae of this strain caused an increase in the size of Müller cell somata to 120.7±2.0% (n=24, P<0.001) (FIG. 3A). Müller cell bodies in slices from control retinas swelled upon hypotonic stress when K⁺ channel-blocking Ba²⁺ ions were present in the bath solution (111.5±1.6%, n=15; P<0.001). By contrast, Müller cells in retinal slices from untreated eyes of Dp71-null mice already displayed osmotic swelling to 118.0±2.3% (n=27, P<0.001 as compared to C57BL/6 Müller cells under the same condition) in the absence of Ba²⁺. Müller cells in the detached retina of Dp71-null mice swelled to an extent that was not significantly different to that of the untreated eye (FIG. 3A; 116.6±2.0%, n=26). Moreover, the application of Ba²⁺ failed to induce an additional swelling in Müller cells from untreated Dp71-null mice (FIG. 3B; 122.3 if 3.6%, n=12).

As it had been shown in a pig model of retinal detachment that osmotic Müller cell swelling can be inhibited by the application of adenosine or of blockers of pathways that produce inflammatory mediators, the nucleoside adenosine (10 μM) was applied with the hypotonic solution onto untreated retinas from Dp71-null mice. Adenosine significantly blocked somatic swelling (103.2±2.2%, n=12; in comparison to 120.3±3.8%, n=8, without adenosine; P<0.01). The enzymes, phospholipase A₂ and cyclooxygenase produce the inflammatory mediators arachidonic acid and prostaglandins, respectively. The selective inhibitors of phospholipase A₂, 4-bromophenacyl bromide (300 μM), and of cyclooxygenase, indomethacin (10 μM), prevented the swelling of Müller cells from Dp71-null mice (FIG. 3C; 103.3±2.1%, n=8, P<0.01 and 104.4±1.6%, n=7, P<0.01, respectively). These data suggest that deletion of Dp71 modifies the osmotic swelling characteristics of Müller cells, and that inflammatory mediators are involved in this alteration.

Dp71 Deletion in MüLler Cells Increases the BRB Permeability:

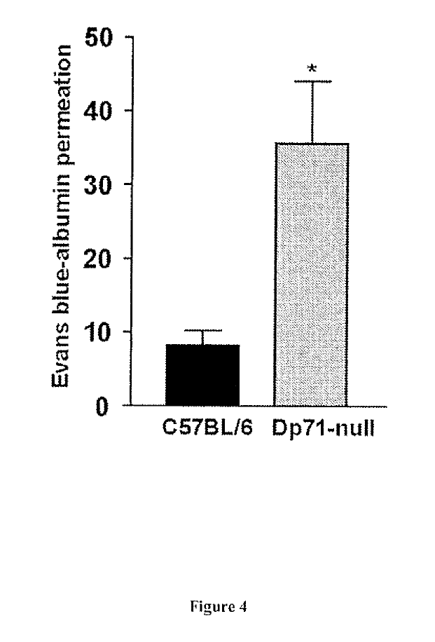

There were two previous observations which prompted us to study the effect of Dp71 depletion on BRB integrity. First, Müller glial cells are in close contact with retinal blood vessels and play an important role in the formation and maintenance of the BRB. Second, Dp71 immunoreactivity is localized in Müller cell endfeet and processes surrounding retinal blood vessels. Retinal vascular permeability was assessed in C57BL/6 and Dp71-null adult mice using the Evans blue-dye technique (Xu Q. et al. 2001). This revealed that the BRB permeability of Dp71-null mice was up to 4-fold elevated (p<0.05) as compared to C57BL/6 mice (FIG. 4). This shows that the absence of Dp71 in Müller cells is accompanied by an increased permeability of the BRB, and may even be indicative of a causal relation between the two features.

Discussion:

Using an experimental mouse model of retinal detachment, we (i) confirm morpho-functional alterations in reactive Müller glial cells including a mislocation of Kir4.1 potassium channels and a downregulation of AQP4 water channels, accompanied by disturbed volume regulation of the cells; (ii) show a fast and strong decrease of the dystrophin protein, Dp71 whereas utrophin, an autosomal paralog of dystrophin, is upregulated; and finally (iii) demonstrate an impaired BRB function as evidenced by increased retinal vascular permeability. These findings indicate a key role for glial Dp71 in the maintenance of retinal potassium and water homeostasis and cellular volume regulation, as well as in the control of the BRB.

In our model of experimental detachment in murine retinas, Müller cells became reactive (as indicated by GFAP expression) and showed a decrease in potassium currents, similar as found in rabbit and porcine models of retinal detachment. Here we show, for the first time, a fast decrease in gene and protein expression of Dp71 (FIG. 1). This may indicate that generally, the mislocation of Kir4.1 channels in reactive Müller cells results from an alteration in Dp71 expression. This hypothesis is supported by earlier data showing that Dp71 is required for proper clustering and precise membrane localization of Kir4.1 and AQP4 channels. Most likely, the function of Dp71 is to stabilize the Kir4.1 and AQP4 channel molecules in specific membrane domains by limiting their lateral diffusion. Inactivation of Dp71 leads to a delocalization of binding partners of Kir4.1 which are involved in the membrane anchoring of this channel. Our present results confirm that functional expression of Dp71 is necessary for the highly asymmetric expression of the inwardly rectifying potassium channel Kir4.1 in Müller cells, and suggest that Dp71 downregulation is one step upstream of the Kir4.1 and AQP4 channel alterations in the signaling cascade of reactive gliosis.

This idea is further supported if the consequences of Dp71 deficiency are studied one step downstream of the channel alterations, by assessing the swelling properties of Müller cells. We observed a similar anomalous osmotic swelling in Müller cells from untreated Dp71-null mice and in cells from C57BL/6 after detachment-induced gliosis (FIG. 3). This finding shows that partial or total depiction of Dp71 in Müller cells are associated with alterations in transmembranous water transport.

In addition to failures in Kir4.1 and AQP4 expression and/or membrane insertion, Dp71 deficiency may also induce a chronic inflammation. We found that similar as in a porcine model of retinal detachment, inflammatory mediators are implicated in osmotic Müller cell swelling in Dp71-null mice. Inhibition of phospholipase A₂ or cyclooxygenase, enzymes producing arachidonic acid and prostaglandins, respectively, prevent the osmotic swelling of Müller cells from Dp71-null mice. We thus suggest that Müller cells of Dp71-null mice undergo a chronic inflammation which contributes to the alterations in their physiological parameters. Indeed, the gene expression of cyclooxygenase-2, known to play a key role in the pathogenesis of inflammation, was up to 7-fold elevated in retinas of Dp71-null mice at postnatal day 6 as compared to C57BL/6. However, there was no significant difference between the cyclooxygenase-2 level of retinae from C57BL/6 and those from Dp71-null adult mice. Further studies are necessary to elucidate the underlying pathomechanisms.

Extracellular accumulation of fluid, resulting in edema, is the most common cause of vision impairment in diabetic retinopathy. Extravascular accumulation of fluid in the subretinal space and the inner retinal tissue is normally prevented by pigment epithelium and Müller cells. Vascular leakage and impaired fluid absorption from the retinal tissue across the glio-vascular interface are major pathogenic events of edema formation. We show here that both pathomechanisms are stimulated by the partial or total absence of Dp71. First, the fluid transport through Müller cell membrane is impaired (FIG. 3) and second, we show here for the first time that Dp71 depletion is accompanied by a significantly increased retinal vascular permeability (FIG. 4). This latter observation is in accordance with previous reports showing that Müller cells participate in the maintenance and the proper functioning of the BRB. In the retina, Dp71 is mainly expressed in Müller cells endfeet and processes surrounding blood vessels. It is thus conceivable that the lack of Dp71 results in morphological and functional alterations of these vitread and perivascular Müller cell compartments and that these impairments lead to a dysregulation of retinal vascular permeability. Presently the underlying molecular signaling cascade of this putative pathomechanism is completely unclear. It may be noteworthy in this context that Müller cells are capable of secreting the vascular endothelial growth factor (VEGF), a key player in increasing the retinal vascular permeability. Obviously, the ‘compensatory’ upregulation of utrophin observed around blood vessels and at the inner limiting membrane is not functionally compensating the absence of Dp71. Under these circumstances, it appears to be surprising that Dp71-null mice do not show impairments in visual functions nor indications of retina degeneration if not challenged. Very probably, the animals develop partial compensatory mechanisms: however, this remains to be proven by the future generation of conditional Dp71 KO mice.

We show here that depletion of Dp71, a cytoskeleton protein associated to the membrane, leads to physiological alterations in Müller cells similar to those observed in injured or diseased retinas; this involves a mislocation of potassium and water channels (Kir4.1 and AQP4) and a consequent dysregulation of water transport through Mailer cells. Furthermore, we show that the early, acute downregulation of Dp71 in normal animals may be an important, decisive step in the development of reactive gliosis, upstream of the physiological alterations. Finally we show the altered Müller cell properties in Dp71-null mice may increase the retinal vascular permeability, via a hitherto unknown mechanism. Taken together, Dp71-null mice appear as a versatile, promising model to develop novel drugs for the resolution of retinal edemas such as observed in human diabetic retinopathy.

### REFERENCES

Throughout this application, various references describe the state of the art to which this invention pertains. The disclosures of these references are hereby incorporated by reference into the present disclosure.
Bermudez de Leon M, Gomez P, Elizondo G, Zatarain-Palacios R, Garcia-Sierra F, Cisneros B. Beta-naphthoflavone represses dystrophin Dp71 expression in hepatic cells. Biochim Biophys Acta. 2006 March-April; 1759 (3-4):152-8. Epub 2006 Apr. 4.
Bordais A, Bolanos-Jimenez F, Fort P, Varela C. Sahel J A, et al. (2005) Molecular cloning and protein expression of Duchenne muscular dystrophy gene products in porcine retina. Neuromuscul Disord 15: 476-487.
Claudepierre T., C. Dalloz, D. Mornet, K. Matsumura, J. Sahel And A. Rendon (2000) Characterization of the intermolecular associations of the dystrophin associated glycoprotein complex in retinal Müller Glial Cells. J. of Cell Science, 113, 3409-3417
Dalloz C, Sarig R, Fort P, Yaffe D, Bordais A, et al. (2003) Targeted inactivation of dystrophin gene product Dp71: phenotypic impact in mouse retina. Hum Mol Genet. 12: 1543-1554.
Fort P E, Serie A, Pannicke T, Roux M J, Forster V, et al. (2008) Kir4.1 and AQP4 associate with Dp71- and utrophin-DAPs complexes in specific and defined microdomains of Müller retinal glial cell membrane. Glia 56: 597-610.
Hugnot, J. P., Gilgenkrantz, H., Vincent, N., Chafey, P., Morris, G. E., Monaco, A. P., Berwald-Netter, Y., Koulakoff, A., Kaplan, J. C., Kahn, A. et al. (1992) Distal transcript of the dystrophin gene initiated from an alternative first exon and encoding a 75-kDa protein widely distributed in nonmuscle tissues. Proc. Nail Acad. Sci. USA, 89, 7506-7510.
J. M. Ervasti, S. D. Kahl and K. P. Campbell, Purification of dystrophin from skeletal muscle, J. Biol. Chem. 266 (1991), pp. 9161-9165.
Kofuji P, Biedermann B, Siddharthan V, Raap M, Iandiev 1, et al. (2002) Kir potassium channel subunit expression in retinal glial cells: implications for spatial potassium buffering. Glia 39: 292-303.
Kramarcy, N. R., Vidal, A., Frochner, S. C. and Scalock, R., 1994. Association of utrophin and multiple dystrophin short forms with the mammalian Mr 58,000 dystrophin-associated protein (syntrophin)*. J Biolog Chem 269, pp. 2870.2876.
Lederfein, D., Levy, Z., Augier, N., Mornet, D., Morris, G., Fuchs, O., Yaffe, D. and Nudel, U., 1992. A 71-kilodalton protein is a major product of the Duchenne muscular dystrophy gene in a brain and other nonmuscle tissues. Proc Natl Acad Sci USA 89, pp. 5346-5350.
O. Ibraghimov-Bcskrovnaya, J. M. Ervasti, C. J. Leveille, C. A. Slaughter, S. W. Semen and K. P. Campbell, Primary structure of dystrophin-associated glycoproteins linking dystrophin to the extracellular matrix, Nature 355 (1992), pp. 696-702.
Rapaport, D., Lederfein, D., den Dunnen, J. T., Grootscholten, P. M., Van Ommen, G. B., Fuchs, O., Nudel, U. and Yaffe, D., 1992. Characterization and cell type distribution of a novel, major transcript of the Duchenne Muscular Dystrophy gene. Differentiation 49, pp. 187-193.
Rivier F, Robert A, Hugon G, Bonet-Kerrache A, Nigro V, et al. (1999) Dystrophin and utrophin complexed with different associated proteins in cardiac Purkinje fibres. Histochem J 31: 425-432.
Sarig R, Mezger-Lallemand V, Gitelman I, Davis C, Fuchs O, et al. (1999) Targeted inactivation of Dp71, the major non-muscle product of the DMD gene: differential activity of the Dp71 promoter during development. Hum Mol Genet. 8: 1-10.
Tomasek J J, Haaksma C J, Schwartz R J, Vuong D T, Zhang S X, Ash J D, Ma J X, Al-Ubaidi M R. Deletion of smooth muscle alpha-actin alters blood-retina barrier permeability and retinal function. Invest Ophthalmol V is Sci. 2006 June:47(6):2693-700.
Xu Q, Qaum T, Adamis A P (2001) Sensitive blood-retinal barrier breakdown quantitation using Evans blue, Invest Ophthalmol V is Sci 42: 789-794.
Yang L, Bula D, Arroyo J G. Chen D F (2004) Preventing retinal detachment-associated photoreceptor cell loss in Bax-deficient mice. Invest Ophthalmol V is Sci 45: 648-654.

1-10. (canceled)

11. A method of screening drugs for treating a disease associated with increased retinal permeability, comprising the steps of
administering to one or more genetically engineered Dp71 null mice one or more candidate compounds;
measuring the effect of said one or more candidate compounds on the blood-retinal barrier integrity of said one or more Dp71 null mice; and
selecting at least one of said one or more candidate compounds which is capable of restoring the blood-retinal barrier integrity in said one or more Dp71 null mice.
